(19) United States
(12) Patent Application Publication
(10) Pub. No.: US 2023/0129707 A1
(43) Pub. Date: Apr. 27, 2023

(54) AUTOMATED SYSTEM AND METHOD FOR ANALYZING SAMPLES FROM A BIOREACTOR

(71) Applicant: Bristol-Myers Squibb Company, Princeton, NJ (US)

(72) Inventors: Letha Chemmalil, Devens, MA (US); Julia Ding, Weston, MA (US); Zhengjian Li, Sudbury, MA (US)

(21) Appl. No.: 17/915,593

(22) PCT Filed: Jun. 29, 2021
(86) PCT No.: PCT/US2021/039640

§ 371 Date: Sep. 29, 2022

Related U.S. Application Data
(60) Provisional application No. 63/045,241, filed on Jun. 29, 2020.

(51) Int. Cl. G01N 30/46; G01N 30/32; G01N 30/10; G01N 30/88; B01D 15/18; C12M 1/34; C12M 1/36

(52) U.S. Cl. CPC G01N 30/463; G01N 30/32; G01N 30/10; G01N 30/88; B01D 15/1878; C12M 41/32; C12M 41/48; G01N 2030/027

(57) ABSTRACT

In one aspect, a method for automated analysis of samples from a bioreactor is provided herein, the method including: drawing at least one sample from a bioreactor; pressurizing the drawn at least one sample into a sample flow; purifying at least one target protein in the sample flow using a first liquid chromatography apparatus to create a purified sample flow; splitting the purified sample flow into a purified sample fraction flow and an effluent flow; and, analyzing the at least one target protein in the purified sample fraction flow using a second liquid chromatography apparatus. Advantageously, the subject invention provides for an automated two-step liquid chromatography process utilizing first dimension liquid chromatography for purification and second dimension liquid chromatography for analysis.

### CROSS-REFERENCE TO RELATED APPLICATIONS

This application is a National Stage Application under 35 U.S.C. § 371 of PCT Application No. PCT/US2021/039640, filed Jun. 29, 2021, which claims the priority benefit of U.S. Provisional Application No. 63/045,241, filed Jun. 29, 2020, the contents of which are herein incorporated by reference in their entireties.

### BACKGROUND OF THE INVENTION

Analyzing samples from a bioreactor using first dimension liquid chromatography for purification and second dimension liquid chromatography for identifying target proteins is known in the prior art. Due to various constraints and sensitivity, second dimension liquid chromatography, however, may only utilize a small fraction of first dimension chromatographic eluate to eliminate the lack of peak resolution associated with large protein peak volumes entering the second dimension liquid chromatography apparatus. In one approach, purified samples are reduced to fractional samples (typically 10%) for use in second dimension liquid chromatography. Compared to the traditional approach, this innovative approach reduces analysis time by ten-fold. Based on the traditional approach, the purified sample may be automatically fractionated to utilize high-resolution peak-cutting, where fractions of purified sample are collected in sample loops to be individually analyzed in second dimension liquid chromatography, with resulting data being conjoined to deliver final results. FIG. 2 is a representative second dimension ion exchange chromatography (IEX) of individual first dimension protein-A peaks resulting from high resolution peak cutting. While this approach works, the analysis time is ten-fold higher than the innovative approach of flow-splitting strategy.

### SUMMARY OF THE INVENTION

In one aspect, an automated system for analyzing at least one sample from a bioreactor is provided herein which includes: a probe for drawing at least one sample from the bioreactor; a pump for pressurizing the drawn at least one sample into a sample flow; a first conduit connected to the pump for conveying the sample flow; a first liquid chromatography apparatus having a primary inlet and a primary outlet, the primary inlet connected to the first conduit to receive the sample flow, the first liquid chromatography apparatus being configured to purify at least one target protein in the sample flow to create a purified sample flow, the purified sample flow being discharged from the primary outlet; a second conduit connected to the primary outlet for conveying the purified sample flow; a flow splitter having a splitter inlet, a branch outlet, and a splitter outlet, the splitter inlet connected to the second conduit to receive the purified sample flow, wherein a flow restrictor is associated with the branch outlet to allow a fraction of the purified sample flow to discharge from the branch outlet as a purified sample fraction flow, and, wherein, the purified sample flow not discharged from the branch outlet is discharged from the splitter outlet as an effluent flow; a third conduit connected to the branch outlet for conveying the purified sample fraction flow; and, a second liquid chromatography apparatus having a secondary inlet connected to the third conduit to receive the purified sample fraction flow, the second liquid chromatography apparatus configured to analyze the at least one target protein in the purified sample fraction flow. Advantageously, the subject invention provides for an automated two-step liquid chromatography process utilizing first dimension liquid chromatography for purification and second dimension liquid chromatography for analysis.

In a further aspect, a method for automated analysis of at least one sample from a bioreactor is provided herein, the method including: drawing at least one sample from a bioreactor; pressurizing the drawn at least one sample into a sample flow; purifying at least one target protein in the sample flow using a first liquid chromatography apparatus to create a purified sample flow; splitting the purified sample flow into a purified sample fraction flow and an effluent flow; and, analyzing the at least one target protein in the purified sample fraction flow using a second liquid chromatography apparatus.

These and other features of the subject invention will be better understood through a study of the following detailed description and accompanying drawings.

### BRIEF DESCRIPTION OF THE DRAWINGS

FIG. 1 is a schematic of a system formed in accordance with the subject invention.

FIG. 2 is a representative second dimension ion exchange chromatography (IEX) of individual first dimension protein-A peaks resulting from high resolution peak cutting.

FIG. 3 is a representative chromatogram of Protein A generated by a system in accordance with the subject invention, using size-exclusion chromatography (SEC).

FIGS. 4 and 5 are representative chromatograms of Protein A generated by the second liquid chromatography apparatus of a system in accordance with the subject invention, using weak and strong cation exchange columns, respectively, with cation exchange chromatography (CEX).

FIG. 6 is a representative profile of online bioreactor titer, measured by the first liquid chromatography apparatus of a system in accordance with the subject invention, taken from day-7 through day-15.

FIG. 7 is a representative online amino acid analysis profile generated using o-Phthaldialdehyde derivatization (OPA) by the first liquid chromatography apparatus of a system in accordance with the subject invention.

FIG. 8 exhibits spike and recovery study results of amino acids spiked in cell culture media, derivatized using o-Phthaldialdehyde derivatization (OPA) in combination with the subject invention versus 6-aminoquinolyl-N-hydroxysuccinimidyl carbamate derivatization (AQC or AQ) in combination with offline processing.

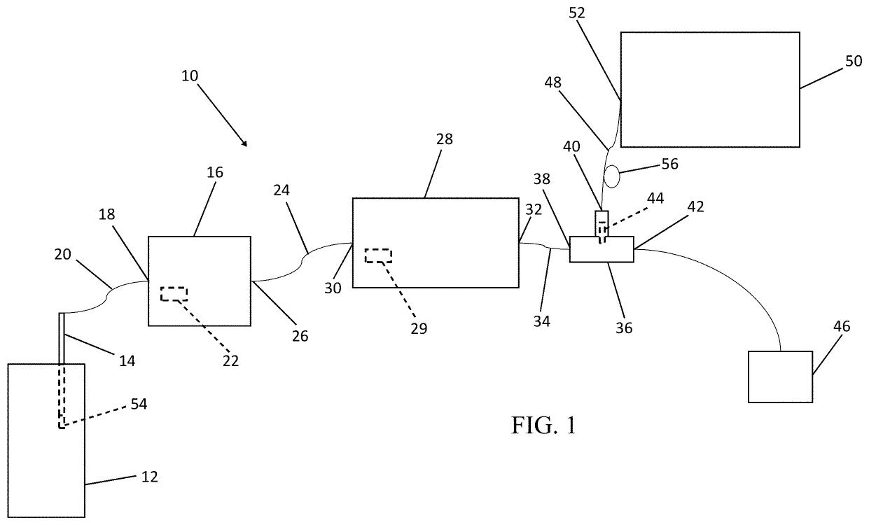

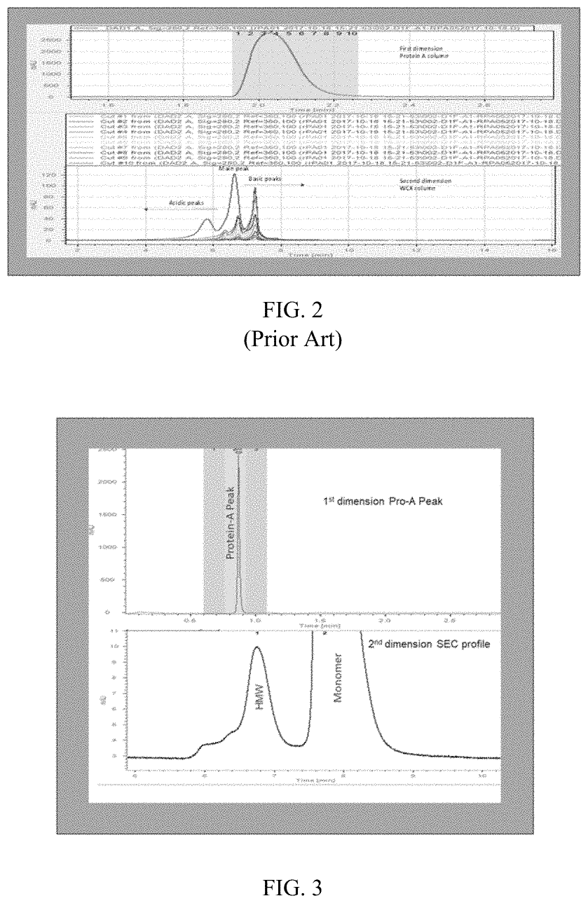

### DETAILED DESCRIPTION OF THE INVENTION

With reference to the Figures, a system 10 is provided for automated analysis of one or more samples taken from a bioreactor 12. The terminology used herein is for the purpose of describing particular embodiments only and is not intended to be limiting of the invention. As used herein, the term “sample,” in singular or plural, covers both cases, without limitation. The bioreactor 12 may be any standard bioreactor for cultivating biological protein samples. A probe 14 may be provided positioned to draw samples from the bioreactor 12. A pump 16 may be provided having an inlet 18 in communication with the probe 14, e.g., via a sampling conduit 20. The pump 16 is configured to generate negative pressure to draw samples from the bioreactor 12 through the probe 14 and the sampling conduit 20.

The pump 16 may be of any known design and may be manifolded to allow for connections with multiple inlet lines in parallel. The pump 16 may be of the peristaltic pump type configured to act on the sampling conduit 20 or a conduit in communication with the sampling conduit 20, without contacting the samples. One or more interim vials 22 may be provided with the pump 16 to collect samples drawn from the bioreactor 12. The samples may be drawn from the interim vials 22 and pressurized by the pump 16 to provide a sample flow out of the pump 16. A first conduit 24 may be provided in communication with an outlet 26 of the pump 16 for conveying the generated sample flow. In addition, a sample collection loop may be associated with the pump 16, the sampling conduit 20, and/or the first conduit 24, to collect samples in preparing the sample flow.

Control system may be provided for controlling the pump 16. The control system may include a computer processing unit, with non-transitory memory for storing instructions. The control system may be configured, e.g., by instructions stored in the memory, to cause activation of the pump 16 at given intervals, or other start times. With automated operation, the pump 16 may act as an autosampler.

A first liquid chromatography apparatus 28 is provided which is preferably configured as a first dimension liquid chromatography apparatus configured to purify at least one target protein in the sample flow in creating a purified sample flow. The first conduit 24 delivers the sample flow to a primary inlet 30 of the first liquid chromatography apparatus 28. The flow rate of the sample flow through the first conduit 24 may be in the range of 0.5-5 mL/min. One or more primary vials 29 may be utilized within the first liquid chromatography apparatus 28 to collect the sample flow for purification. Any known design of liquid chromatography apparatus may be utilized suitable for purifying target proteins in a sample flow.

The first liquid chromatography apparatus 28 includes a primary outlet 32 to which is connected to a second conduit 34. A purified sample flow is discharged from the first liquid chromatography apparatus 28 through the primary outlet 32 into the second conduit 34. The purified sample flow may be discharged as a volume in the range of 1-100 μl, alternatively in the range of 1-80 μl, alternatively in the range of 1-60 μl, alternatively in the range of 1-40 μl, alternatively 1-20 μl, and alternatively 1-10 μl. The first liquid chromatography apparatus 28 may include a pump for pressurizing the discharged purified sample flow and for drawing the sample from the primary vials 29, if utilized. The purified sample flow includes an increased percentage of the at least one target protein which was purified by the first liquid chromatography apparatus 28.

A flow splitter 36 is provided for the system 10 having a splitter inlet 38, a branch outlet 40, and a splitter outlet 42. The splitter inlet 38 is connected to the second conduit 34 to receive the purified sample flow. A flow restrictor 44 is associated with the branch outlet 40 such that only a fraction of the purified sample flow is permitted to discharge from the branch outlet 40 as a purified sample fraction flow. The purified sample fraction flow may be any portion of the purified sample flow, including being no greater than 50% of the purified sample flow entering the splitter inlet 38, alternatively being no greater than 33.3% of the purified sample flow entering the splitter inlet 38, and alternatively being no greater than 10% of the purified sample flow entering the splitter inlet 38. The purified sample fraction flow may represent a volume in the range of <40 μl. Any portion of the purified sample flow not discharged from the branch outlet 40 is discharged from the splitter outlet 42 as an effluent flow which can be collected in a waste receptacle 46. As will be appreciated by those skilled in the art, the flow restrictor 44 may be located in the splitter outlet 42, or, alternatively, a plurality of flow restrictors 44 are utilized located in one or both of the branch outlet 40 and the splitter outlet 42.

A third conduit 48 is connected to the branch outlet 40 to convey the purified sample fraction flow.

A second liquid chromatography apparatus 50 is provided having a secondary inlet 52 connected to the third conduit 48 to receive the purified sample fraction flow. The second liquid chromatography apparatus 50 is configured to analyze the one or more target proteins in the purified sample fraction flow. Any known design of liquid chromatography apparatus may be utilized suitable for analyzing the target proteins, including second dimension liquid chromatography apparatuses. Due to sensitivity and other constraints, the size of samples for analysis by the second liquid chromatography apparatus 50 is limited. The flow splitter 36 allows for automated diverting of a fraction of the purified sample flow to the second liquid chromatography apparatus 50 for analysis thereby.

The third conduit 48 and/or the second liquid chromatography apparatus 50 may be provided with at least one sample collection loop 56 for collecting the purified sample fraction flow to amass a certain volume for an injection in the second liquid chromatography apparatus 50. If a plurality is utilized, the sample collection loops 56 may be arranged in parallel to sequentially collect the purified sample fraction flow. The sample collection loop(s) 56 allow for smaller volume flow rate to be discharged from the branch outlet. For example, the flow splitter 36 may be configured to discharge 10% of the purified sample flow entering the splitter inlet 38 as purified sample fraction flow. The purified sample fraction flow may collect in a single sample collection loop 56 to a pre-determined volume, such as 40 μl, ready for injection. With a plurality, the sample collection loops 56 may collectively collect the purified sample fraction flow to the pre-determined volume. The purified sample fraction flow may be drawn into the second liquid chromatography apparatus 50 and/or the sample collection loop(s) 56 by a pump located in the second liquid chromatography apparatus 50 and/or located along the third conduit 48. The pump may be automated to actuate with the pre-determined volume being detected in the sample collection loop(s) 56.

Optionally, a filter 54 may be introduced on the probe 14 and/or the sampling conduit 20 for filtering samples drawn from the bioreactor 12.

Standard cleaning techniques may be utilized between analyses, including running the system 10 through one or more operational cycles utilizing a cleaning solution.

Certain components of the system 10 may need to be replaced after a certain number of cycles, such as the probe 14, one or more of the conduits, and/or portions of the flow splitter 36.

As will be appreciated by those skilled in the art, the system 10 may be utilized, for example, for purification and analysis of monoclonal antibodies (mAbs) and Fc fusion proteins.

By way of non-limiting example, the system 10 may include: the first liquid chromatography apparatus 28 may be any 1D-LC commercially available liquid chromatography apparatus capable of purifying a target sample, such as, a liquid chromatography apparatus sold under the name “1260 Infinity” or “1290 Infinity” by Agilent Technologies, Inc. of Santa Clara, Calif.; the second liquid chromatography apparatus 50 may be any 2D-LC commercially available liquid chromatography apparatus capable of analyzing a purified sample flow, such as a liquid chromatography apparatus sold under the name “1290 Infinity” by Agilent Technologies, Inc. of Santa Clara, Calif.; the pump 16 may be any commercially available automated on-line sampling system, such as sold under the name “Seg-Flow 4800” by Flownamics Analytical Instruments, Inc. of Madison, Wis.; and, the flow splitter 36 may be any commercially available flow splitter, such as sold under the name “PerfectPeak” by Mott Corporation of Farmington, Conn.

The subject invention allows for continuous bioprocessing, allowing for near-real-time monitoring of titer levels, and critical quality attributes (CQAs) of processed samples, as well as, amino acid levels in the bioreactor 12. For example, the first liquid chromatography apparatus 28 may be configured to analyze amino acid levels in the sample flow delivered by the first conduit 24. In this manner, feedback control may be established to add depleted amino acids back into the bioreactor 12.

In addition, the first liquid chromatography apparatus 28 and/or the second liquid chromatography apparatus 50 may be configured to analyze titer levels in the processed sample, as well as, CQAs.

### Examples

Utilizing the system 10, online size and charge variant analysis results, generated using protein A chromatography in the first dimension followed by 1:10 flow splitting prior to second dimension size-exclusion chromatography (SEC) and cation exchange chromatography (CEX) analyses, showed that the system 10 worked well with results comparable to the results generated using offline test results. Tables 1 and 2 show comparative data of SEC and CEX chromatography using the system 10 (online) and offline (manual) processing. Samples 1-4 are mAb molecules taken from a bioreactor, analyzed on different days.

TABLE 1
Online vs. Offline SEC data (% purity)

| Mode of | | | | |
|---|---|---|---|---|
| analysis | Sample 1 | Sample 2 | Sample 3 | Sample 4 |
| SEC Online | 94.6% | 95.1% | 94.3% | 95.5% |
| SEC Offline | 94.5% | 95.4% | 94.8% | 95.2% |

TABLE 2
Online vs. Offline CEX data (% Main)

| Mode of | | | | |
|---|---|---|---|---|
| analysis | Sample 1 | Sample 2 | Sample 3 | Sample 4 |
| CEX Online | 58.0% | 67.4% | 70.5% | 69.6% |
| CEX Offline | 67.8% | 69.2% | 68.3% | 67.2% |

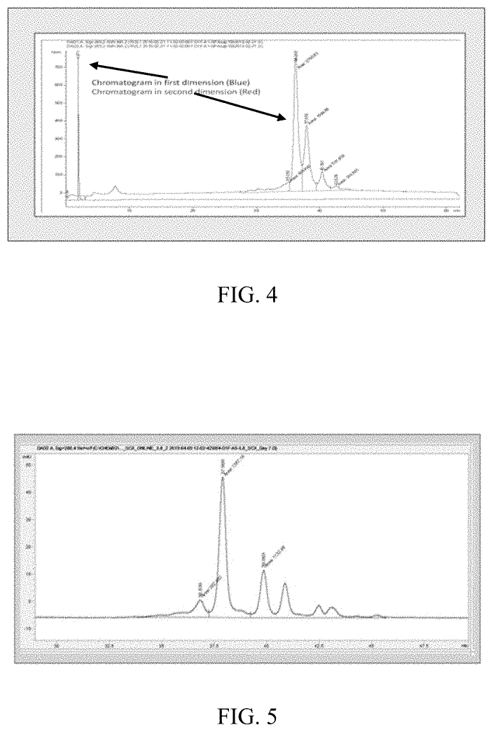

A representative chromatogram of Protein A/SEC generated by the system 10 is shown in FIG. 3. Representative protein A/CEX chromatograms generated by the second liquid chromatography apparatus 50 using weak and strong cation exchange columns are shown in FIGS. 4 and 5, respectively.

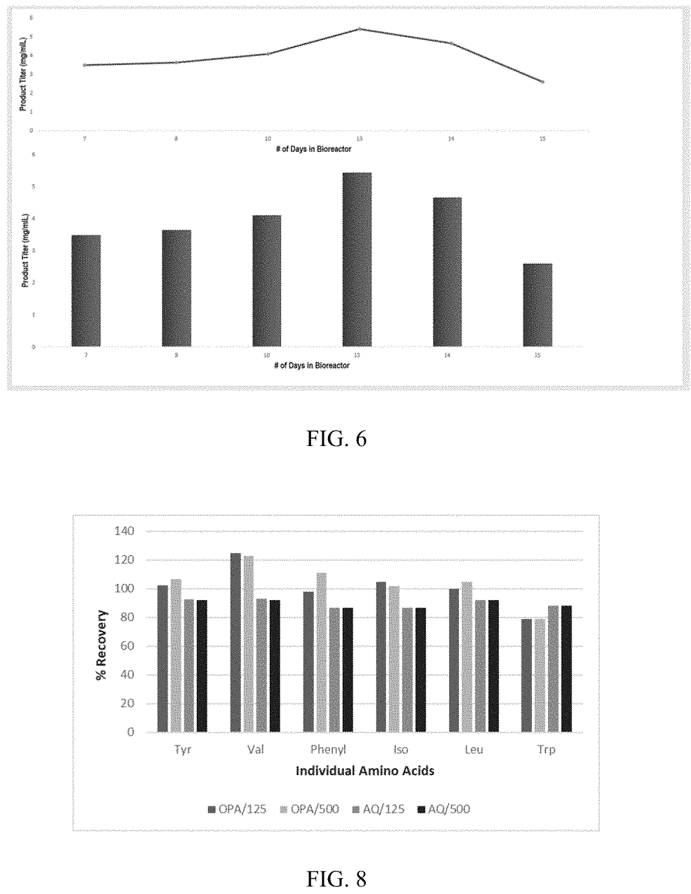

In addition to the product quality results, concurrently generated titer results from the first dimension protein-A chromatography is an added advantage. A representative profile of online bioreactor titer, measured by the first liquid chromatography apparatus 28, from day-7 through day-15 is depicted in FIG. 6. Other potential chromatographic techniques such as RP-HPLC, HIC, HILIC, affinity chromatography and denaturing SEC (reduced & non-reduced) can also be performed for bioreactor samples using the system 10.

### Amino Acid Analysis (AAA)

The system 10 may be used in connection with in-column o-Phthaldialdehyde derivatization (OPA) and inline sampling, thereby enabling the system 10 to be fully automated to do online amino acid analysis (AAA). A representative online amino acid analysis profile generated using the first liquid chromatography apparatus 28 with OPA, interfaced with the pump 16 functioning as an autosampler, is shown in FIG. 7.

A comparison of OPA, using the subject invention, versus 6-aminoquinolyl-N-hydroxysuccinimidyl carbamate derivatization (AQC or AQ) (e.g., AccQ-Tag sold by Waters Corporation, Milford, Mass., USA) using an offline (manual) combination of first and second dimension chromatography, shows that the apparent differences in results between the two methods are within the inherent variability of AAA. For example, Tables 3 and 4 show results of spike and recovery studies performed by spiking amino acids in NAOH, demonstrating satisfactory recovery achieved for both methods (OPA/subject invention versus AQC/offline combination).

TABLE 3
% recovery of amino acids spiked in NaOH

| | AQC method | OPA method | AQC method | OPA method |
|---|---|---|---|---|
| Amino Acid | (500 mM spike) | (125 mM spike) | (500 mM spike) | (125 mM spike) |
| Tyrosine | 95.7% | 98.2% | 102.6% | 114.3% |
| Valine | 101.9% | 102.5% | 108.2% | 118.2% |
| Iso-Leucine | 95.3% | 101.2% | 101.0% | 116.6% |
| Leucine | 101.3% | 109.2% | 107.7% | 125.5% |
| Phenylalanine | 90.3% | 93.4% | 97.4% | 109.4% |
| Tryptophan | 90.7% | 92.8% | 96.6% | 107.6% |

TABLE 4
Comparability study results of spike &recovery studies of known
amount (μM) of Amino acids spiked in NaOH &cell culture media

| Sample Name | Tyr (uM) | Val (uM) | Ile (uM) | Leu (uM) | Phe (uM) | Trp (uM) |
|---|---|---|---|---|---|---|
| Media 500/AccQ | 297.4 | 277.8 | 216.9 | 231.8 | 276.3 | 296.8 |
| Media 500/OPA | 260.7 | 188 | 188.6 | 205.4 | 243.5 | 271.1 |
| NaOH 500/AccQ | 488.6 | 509.4 | 476.3 | 506.4 | 451.5 | 453.5 |
| NaOH 500/OPA | 491.1 | 512.7 | 506 | 546 | 466.8 | 464 |
| NaOH-125/AccQ | 128.2 | 135.3 | 126.3 | 134.6 | 121.7 | 120.7 |
| NaOH-125/OPA | 142.8 | 147.7 | 145.8 | 158.1 | 136.8 | 134.5 |

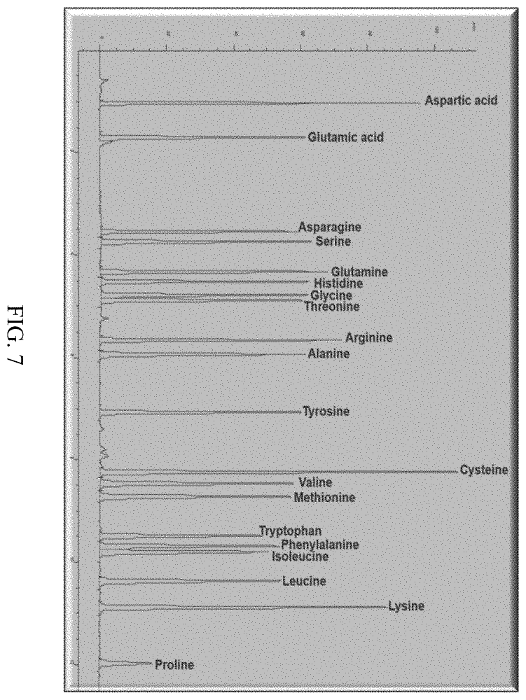

FIG. 8 exhibits the spike and recovery study results of amino acids spiked in cell culture media, demonstrating adequate comparatively with better recovery for the OPA/subject invention versus AQC/offline. A representative online AAA profile of each amino acid during a bioreactor run from day-7 through day-16 is presented in Table 5, utilizing OPA/subject invention.

TABLE 5
Online AAA results of a typical bioreactor run using in-column OPA derivatization

| Days | Asp | Glu | Asn | Ser | Arg | Ala | Tyr | Cys | NorVal | Trpt | Phy | ile | Leu | Lys |
|---|---|---|---|---|---|---|---|---|---|---|---|---|---|---|
| 7 | 4.1 | 11.5 | 5.9 | 14.7 | 4.1 | 5.3 | 5.3 | 6.0 | 3.2 | 3.7 | 2.7 | 11.2 | 12.2 | 4.6 |
| 8 | 4.2 | 11.7 | 2.8 | 18.4 | 2.9 | 2.1 | 2.1 | 6.7 | 3.0 | 2.7 | 2.1 | 12.1 | 9.0 | 4.5 |
| 10 | 4.6 | 11.1 | 6.0 | 15.9 | 3.0 | 1.5 | 1.5 | 7.5 | 2.1 | 5.4 | ND | 10.9 | 6.2 | 4.6 |
| 14 | 4.7 | 10.2 | 6.3 | 14.9 | 3.6 | 2.4 | 2.4 | 11.5 | ND | ND | ND | 11.9 | 7.2 | 5.4 |
| 15 | 4.4 | 10.4 | 5.4 | 14.6 | 4.2 | 2.3 | 11.1 | 13.1 | 4.0 | ND | 1.5 | 5.7 | 5.6 | 5.8 |
| 16 | 4.8 | 8.7 | 3.5 | 16.1 | 4.5 | 2.4 | 9.4 | 13.9 | 4.0 | ND | ND | 6.9 | 6.4 | 5.8 |

What is claimed is:

1. An automated system for analyzing at least one sample from a bioreactor, the system comprising:
a probe for drawing the at least one sample from the bioreactor;
a pump for pressurizing the drawn at least one sample into a sample flow;
a first conduit connected to the pump for conveying the sample flow;
a first liquid chromatography apparatus having a primary inlet and a primary outlet, the primary inlet connected to the first conduit to receive the sample flow, the first liquid chromatography apparatus being configured to purify at least one target protein in the sample flow to create a purified sample flow, the purified sample flow being discharged from the primary outlet;
a second conduit connected to the primary outlet for conveying the purified sample flow;
a flow splitter having a splitter inlet, a branch outlet, and a splitter outlet, the splitter inlet connected to the second conduit to receive the purified sample flow, wherein a flow restrictor is associated with the branch outlet to allow a fraction of the purified sample flow to discharge from the branch outlet as a purified sample fraction flow, and, wherein, the purified sample flow not discharged from the branch outlet being discharged from the splitter outlet as an effluent flow;
a third conduit connected to the branch outlet for conveying the purified sample fraction flow; and,
a second liquid chromatography apparatus having a secondary inlet connected to the third conduit to receive the purified sample fraction flow, the second liquid chromatography apparatus configured to analyze the at least one target protein in the purified sample fraction flow.

2. A system as in claim 1, wherein the drawn at least one sample is collected from the bioreactor in one or more interim vials.

3. A system as in claim 2, wherein the pump draws the drawn at least one sample from the one or more interim vials and, then, pressurizes the drawn at least one sample into the sample flow.

4. A system as in claim 3, wherein the first liquid chromatography apparatus includes one or more primary vials which collect the sample flow.

5. A system as in claim 1, wherein the first liquid chromatography apparatus is a first-dimension liquid chromatography apparatus.

6. A system as in claim 1, further comprising a filter for filtering the at least one sample prior to being pressurized by the pump.

7. A system as in claim 1, wherein the purified sample fraction flow is no greater than 50% of the purified sample flow.

8. A system as in claim 1, further comprising at least one sample collection loop for collecting the purified sample fraction flow to amass a pre-determined volume for analysis by the second liquid chromatography apparatus.

9. A method for automated analysis of at least one sample from a bioreactor, the method comprising:
drawing at least one sample from a bioreactor;
pressurizing the drawn at least one sample into a sample flow;
purifying at least one target protein in the sample flow using a first liquid chromatography apparatus to create a purified sample flow;
splitting the purified sample flow into a purified sample fraction flow and an effluent flow; and,
analyzing the at least one target protein in the purified sample fraction flow using a second liquid chromatography apparatus.

10. A method as in claim 9, wherein the drawn at least one sample is collected from the bioreactor in one or more interim vials.

11. A method as in claim 10, wherein a pump draws the drawn at least one sample from the one or more interim vials and, then, pressurizes the drawn at least one sample into the sample flow.

12. A method as in claim 11, wherein the first liquid chromatography apparatus includes one or more primary vials which collect the sample flow.

13. A method as in claim 9, wherein the first liquid chromatography apparatus is a first-dimension liquid chromatography apparatus.

14. A method as in claim 9, further comprising filtering the drawn at least one sample prior to being pressurized.

15. A method as in claim 9, wherein the purified sample fraction flow is no greater than 50% of the purified sample flow.

16. A method as in claim 9, wherein the at least one sample includes one or more monoclonal antibodies.

17. A method as in claim 9, wherein the at least one sample includes one or more Fc fusion proteins.

18. A method as in claim 9, further comprising collecting the purified sample fraction flow to amass a pre-determined volume, prior to analyzing the at least one target protein in the purified sample fraction flow using the second liquid chromatography apparatus.

19. A method as in claim 18, wherein the purified sample fraction flow is collected in at least one collection sample loop.
